(19) United States
(12) Patent Application Publication
(10) Pub. No.: US 2011/0319693 A1
(43) Pub. Date: Dec. 29, 2011

(54) IONIC LIQUID CATALYZED ALKYLATION WITH ETHYLENE IN ETHYLENE CONTAINING GAS STREAMS

(75) Inventors: Sven Ivar Hommeltoft, Pleasant Hill, CA (US); Hye-Kyung C. Timken, Albany, CA (US)

(73) Assignee: Chevron U.S.A. Inc., San Ramon, CA (US)

(21) Appl. No.: 13/161,737

(22) Filed: Jun. 16, 2011

Related U.S. Application Data
(60) Provisional application No. 61/359,739, filed on Jun. 29, 2010.

(51) Int. Cl. C07C 2/60

(52) U.S. Cl. 585/711

(57) ABSTRACT

An alkylation process comprising contacting in an alkylation zone under alkylation conditions an olefin containing gas stream with an isoparaffin in the presence of an ionic liquid catalyst composition to provide an alkylate product. In an embodiment, the olefin stream may comprise offgas containing ethylene together with one or more non-condensable and/or inert gases, and the offgas may be fed in its native state to an alkylation reactor containing the ionic liquid catalyst for the alkylation of isoparaffins to provide low volatility, high octane gasoline blending components.

This application claims the benefit of U.S. Provisional Application No. 61/359,739 filed on Jun. 29, 2010.

### FIELD OF THE INVENTION

The present invention relates to alkylation processes using ethylene containing gas streams.

### BACKGROUND OF THE INVENTION

Due to increased supply and decreased demand, isopentane is abundantly available in modern refineries. Conventional processes for alkylation of isopentane with olefins have used large quantities of potentially hazardous concentrated sulfuric and hydrofluoric acids as catalyst. These conventional catalysts are, however, ineffective in the alkylation of isoparaffins, such as isopentane, with ethylene.

Ionic liquids may be used as catalysts in various reactions, including isoparaffin alkylation. U.S. Pat. No. 5,750,455 to Chauvin et al. discloses alkylation with olefins in the presence of an ionic liquid and a copper compound. U.S. Pat. No. 6,028,024 to Hirschauer et al. discloses alkylation with olefins in the presence of an ionic liquid and a Group IVB metal compound. U.S. Pat. No. 7,432,408 to Timken et al. discloses a process for alkylating isoparaffins using an ethylene-enriched gas from an ethylene extraction unit. However, ethylene enrichment, e.g., via cryogenic distillation, is costly.

There is a need for more efficient alkylation processes that consume excess and/or low value feedstocks in the production of high value alkylate product using environmentally friendly and highly effective catalysts.

### SUMMARY OF THE INVENTION

According to one aspect of the present invention there is provided an alkylation process comprising contacting, in an alkylation zone under alkylation conditions, an olefin containing gas stream containing not more than about 45 vol % olefins with an isoparaffin in the presence of an ionic liquid catalyst composition to provide an alkylate product.

According to another aspect of the present invention there is provided an alkylation process comprising contacting, in an alkylation zone under alkylation conditions, an ethylene containing gas stream containing not more than about 45 vol % ethylene with an isoparaffin in the presence of a catalyst composition to provide an ethylene conversion of at least about 65%.

According to a further aspect of the present invention there is provided an alkylation process comprising feeding an olefin containing native offgas into an alkylation zone in the presence of an ionic liquid catalyst composition, wherein the offgas contains not more than about 45 vol % olefins; and contacting an isoparaffin with the ionic liquid catalyst composition in the alkylation zone under alkylation conditions to provide an olefin conversion of at least about 65%.

### BRIEF DESCRIPTION OF THE DRAWINGS

FIG. 1 schematically represents an alkylation process and system, according to one aspect of the present invention; and

FIG. 2 is a graph showing ethylene conversion during ionic liquid catalyzed isoparaffin alkylation using a dilute ethylene containing stream, according to another aspect of the present invention.

### DETAILED DESCRIPTION OF THE INVENTION

The present invention provides new and improved processes that use ionic liquid catalysts for the alkylation of isoparaffins, such as isopentane, with olefins, such as ethylene. Using highly effective, and yet environmentally friendly, ionic liquid catalysts, the present invention enables the alkylation of isoparaffins, such as isopentane, by the direct injection of a dilute olefin-containing gas to an alkylation zone or reactor containing an ionic liquid catalyst. As a non-limiting example, the olefin-containing gas may comprise native refinery offgas, such as unprocessed ethylene-containing offgas from a fluidic catalytic cracking (FCC) unit.

One advantage of alkylation processes of the present invention is the elimination of large volumes of potentially hazardous concentrated mineral acids (HF and H₂SO₄). Another advantage of processes of the present invention is the use of more active and selective ionic liquid catalysts. Still a further advantage of processes of the present invention is the elimination of the prior art requirement for the costly cryogenic separation of olefin-containing gas streams to provide an ethylene-enriched fraction. Accordingly, the present invention allows the production of high value, low volatility gasoline blending components with increased efficiency and at lower cost.

In an embodiment, the present invention provides processes for alkylating isoparaffins using olefins such as ethylene. Such processes provide alkylate products useful as gasoline blending components. In an embodiment, processes of the present invention convert undesirable or low value isopentane to high value gasoline blending components, such as dimethyl pentane and trimethylbutane, by alkylation of the isopentane with ethylene from an ethylene-containing refinery stream. Such processes may be performed in an alkylation zone under alkylation conditions in the presence of an ionic liquid catalyst, such as a chloroaluminate ionic liquid. In a sub-embodiment, olefins other than ethylene, such as propylene, butylenes, and pentenes, may also be used for the alkylation of isopentane to make valuable alkylate product. Advantageously, the present invention uses hydrocarbon materials, such as isopentane, that may be present at refineries in excess, thereby reducing or eliminating concerns over the storage and usage of such materials.

The present invention also solves problems associated with excess fuel gas production, for example, by using ethylene in an unprocessed olefin-containing gas stream for isoparaffin alkylation processes. According to one aspect of the present invention, an olefin containing gas stream useful for isoparaffin alkylation may be relatively dilute with respect to its olefin (e.g., ethylene) content. For example, in an embodiment, the olefin containing gas stream may generally contain not more than about 45 vol % olefins, in some embodiments not more than about 35 vol % olefins, in other embodiments not more than about 25 vol % olefins, in a sub-embodiment not more than about 20 vol % olefins, and in another sub-embodiment not more than about 15 vol % olefins.

According to one aspect of the present invention, the olefin containing gas stream may comprise offgas, such as offgas from a refinery process. In an embodiment, such offgas may generally contain not more than about 45 vol % ethylene, in some embodiments not more than about 35 vol % ethylene, in other embodiments not more than about 25 vol % ethylene, in a sub-embodiment not more than about 20 vol % ethylene, and in another sub-embodiment not more than about 15 vol % ethylene. In an embodiment, the olefin containing gas stream may comprise offgas from a FCC unit. That is to say, such offgas may be used as a source of one or more olefins, including ethylene, for the alkylation of isoparaffins, such as isopentane. Refinery offgas, such as FCC unit offgas may also contain substantial amounts of various other gases, such as hydrogen, methane, and nitrogen, as well as ethylene. Other olefin streams containing ethylene, such as coker gas, may also be used in practicing the present invention.

By using offgas for the alkylation of isoparaffins, such as excess refinery isopentane, the overall volume of gasoline produced per unit of crude is increased. In addition, the net amount of fuel gas from the FCC de-ethanizer can be reduced, thus lowering the burden of fuel gas processing equipment. A further benefit of the present invention is that the expensive step of ethylene enrichment of dilute olefin streams (e.g., FCC offgas) may be avoided or eliminated.

Processes of the present invention, allow the direct utilization of both dilute olefin-containing gas streams and excess quantities of isopentane. Additionally, the present invention also allows the use of more conventional alkylation feed components, such as butene, propylene, pentene and isobutane, to produce high quality gasoline blending components. These processes harness the high activity and selectivity of ionic liquid catalysts disclosed herein, such as alkyl substituted pyridinium and imidazolium chloroaluminates. Alkylation processes using chloroaluminate ionic liquid catalysts are disclosed, for example, in commonly owned U.S. Pat. No. 7,531,707 to Harris et al., the disclosure of which is incorporated by reference herein in its entirety.

According to an embodiment of the present invention, an olefin-containing refinery stream may be used as a feedstock for isoparaffin alkylation. Examples of such streams include, without limitation, FCC offgas, coker gas, olefin metathesis unit offgas, polyolefin gasoline unit offgas, and methanol to olefin unit offgas. In an embodiment, an olefin for use in processes of the present invention comprises ethylene. A convenient source of ethylene for conducting a process according to the present invention is native offgas from an FCC unit. Typically, the olefin containing offgas or gas stream for use with the present invention may contain ethylene at concentrations substantially as described hereinabove. Such offgas or gas streams may also contain olefins other than ethylene, such as propylene, butylenes and pentenes.

Another feedstock for processes of the present invention is a refinery stream which contains isoparaffins, notably isopentane. Refinery streams which contain isopentane and which may be used in processes of the present invention include, but are not limited to, extracted isopentane from an FCC unit, a hydrocracking unit, C₅ and C₆ streams from crude unit distillation, and extracted C₅ and C₆ streams from a reformer. An isoparaffin-containing stream for use with the present invention may also contain other isoparaffins such as isobutane. Isobutane may be obtained, for example, from hydrocracking units or may be purchased.

Ionic liquid catalysts that may be useful in practicing the present invention may comprise, for example, a chloroaluminate ionic liquid prepared from a metal halide and an organic halide salt. The metal halide may be, for example, AlCl₃. The preparation of chloroaluminate ionic liquid catalysts is described in commonly owned U.S. Pat. No. 7,495,144 to Elomari, the disclosure of which is incorporated by reference herein in its entirety.

Examples of ionic liquid catalysts that may be useful in practicing the present invention include those prepared from AlCl₃ and an organic halide salt of the general formulas A, B, C, and D:

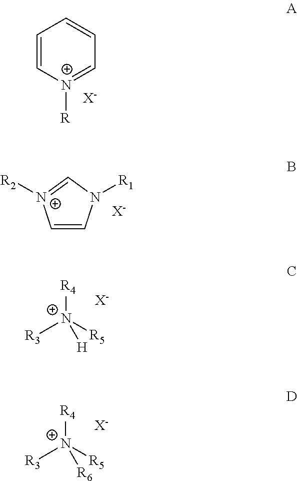

wherein R═H, methyl, ethyl, propyl, butyl, pentyl or hexyl, X is halide, each of R₁ and R₂═H, methyl, ethyl, propyl, butyl, pentyl or hexyl, wherein R₁ and R₂ may or may not be the same, each of R₃, R₄, R₅ and R₆=methyl, ethyl, propyl, butyl, pentyl or hexyl, wherein R₃, R₄, R₅ and R₆ may or may not be the same. An exemplary ionic liquid catalyst that may be use for alkylation of isoparaffins with ethylene from a dilute olefin containing stream is 1-butylpyridinium heptachlorodialuminate, Formula I.

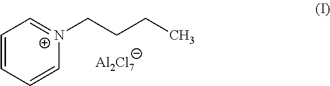

Processes of the present invention may be performed with or without a metal halide co-catalyst, such as NaCl, LiCl, KCl, BeCl₂, CaCl₂, BaCl₂, SiCl₂, MgCl₂, CuCl, AgCl, and PbCl₂ (see, for example, Roebuck and Evering, Ind. Eng. Chem. Prod. Res. Develop., Vol. 9, 77, 1970), as well as Group IVB metal halides (see, for example, U.S. Pat. No. 6,028,024 to Hirschauer et al.).

HCl may also be used as a co-catalyst. The use of HCl as a co-catalyst with 1-butylpyridinium chloroaluminate ionic liquid for ethylene alkylation with isopentane is demonstrated in commonly owned U.S. Pat. No. 7,432,408, the disclosure of which is incorporated by reference herein in its entirety.

Like most reactions in ionic liquids, alkylation according to the present invention is generally biphasic and takes place at the interface in the liquid state. The catalytic alkylation reaction may be performed in a liquid hydrocarbon phase using a batch system, a semi-batch system or a continuous system, with one reaction stage. The isoparaffin(s) and olefins) can be introduced into the alkylation zone either separately or as a mixture. The isoparaffin/olefin molar ratio is typically in the range from about 1 to 100, for example, advantageously in the range from about 2 to 50, and often in the range from about 2 to 20. In a semi-batch system the isoparaffin is introduced first then the olefin, or a mixture of isoparaffin and olefin. Catalyst volume in the reactor is typically in the range from about 2 vol % to 70 vol %, and usually from about 5 vol % to 50 vol %. Vigorous stirring may be used to ensure good contact between the reactants and the ionic liquid catalyst.

The reaction temperature may typically be in the range from about −40° C. to +150° C., and usually from about −20° C. to +100° C. The pressure can be in the range from atmospheric pressure to about 8000 kPa, and is typically sufficient to keep the reactants in the liquid phase. Residence time of reactants in the vessel may be in the range of a few seconds to hours, and typically from about 0.5 min to 60 min. The heat generated by the reaction can be eliminated by any means known in the art. At the reactor outlet, the hydrocarbon phase may be separated from the ionic liquid phase, the hydrocarbons separated by distillation, and any unconverted isoparaffin(s) recycled to the reactor.

Typical reaction conditions may include a catalyst volume in the reactor of from about 5 vol % to 50 vol %, a temperature of from about −10° C. to 100° C., a pressure from about 300 kPa to 2500 kPa, an isoparaffin to olefin molar ratio from about 2 to 8, and a residence time from about 1 min to 1 hour.

As a non-limiting example, a catalyst system or composition for the alkylation of isoparaffins using dilute olefin streams according to the invention, may comprise a chloroaluminate ionic liquid in combination with a HCl co-catalyst. The use of HCl as a co-catalyst may enhance the reaction rate, e.g., by a factor of >6 under comparable conditions, with comparable product selectivity. A catalyst composition of the invention may further include an alkyl halide promoter.

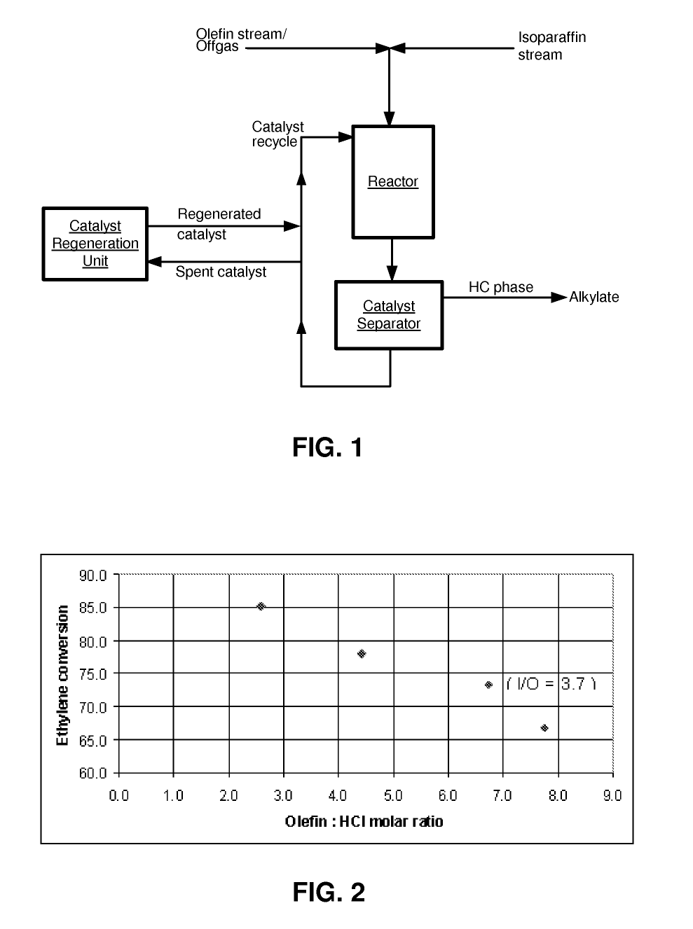

A process and system for the alkylation of isoparaffins using olefin-containing gas streams, according to an embodiment of the present invention, is schematically represented in FIG. 1. The olefin-containing gas stream may comprise a mixture of one or more olefins with one or more other components, such as one or more non-condensable and/or inert gases. The term “non-condensable gas” as used herein refers to a gaseous material, such as may be derived from chemical or petroleum processing, that is not readily condensed by cooling at typical refinery conditions. Examples of such gases include nitrogen, methane, hydrogen, and carbon dioxide.

In an embodiment, the olefin-containing gas stream may comprise a refinery gas stream. For example, in an embodiment, the olefin-containing gas stream may comprise offgas from a refinery upgrading unit, such as a fluidic catalytic cracking (FCC) unit. As a non-limiting example, the olefin-containing gas stream may comprise native, or raw, offgas from an FCC unit. The terms “native offgas” and “raw offgas” as used herein are synonymous and may be used interchangeably. The term “offgas” may be used herein to refer to gaseous material produced as a side effect during one or more petroleum refining or chemical processes. By “native offgas” is meant offgas derived from a process, such as fluidic catalytic cracking, wherein the offgas has not been treated or processed, e.g., in a manner to enrich the offgas in one or more olefin components.

In an embodiment, the olefin-containing gas stream may contain ethylene. The olefin-containing gas stream will generally contain not more than about 45 vol % ethylene. In some embodiments, such an ethylene-containing stream may contain not more than about 35 vol %, not more than about 25 vol %, not more about 20 vol %, or not more than about 15 vol % ethylene. In an embodiment, the olefin-containing gas stream may contain one or more non-condensable gases, such as methane, hydrogen, or mixtures thereof. In another embodiment, the olefin-containing gas stream may contain one or more inert gases, such as nitrogen. In an embodiment, the olefin-containing gas stream may contain at least about 50 vol %, in some embodiments at least about 55 vol %, of one or more gases comprising nitrogen, methane, and hydrogen, or mixtures thereof.

With further reference to FIG. 1, the olefin-containing gas stream or offgas, such as native offgas from an FCC unit, may be fed to an alkylation zone (reactor). According to one aspect of the present invention, the olefin-containing gas stream contains ethylene, and the ethylene-containing gas may be fed or injected directly into a catalyst composition in the reactor, wherein the catalyst composition may comprise a chloroaluminate ionic liquid catalyst.

A second, isoparaffin stream is also fed to the reactor (alkylation zone). The olefin stream and the isoparaffin stream may be introduced separately into the reactor, or the olefin and isoparaffin stream may be combined prior to their introduction into the reactor. In the reactor, the olefin and isoparaffin streams may be contacted in the presence of the ionic liquid catalyst under alkylation conditions to provide an alkylate product. The isoparaffin stream may comprise, for example, isopentane, isobutane, or mixtures thereof. Typically, isopentane is abundantly available in modern refineries from various upgrading processes, such as fluidic catalytic cracking, hydrocracking, and paraffin isomerization. In an embodiment, the isoparaffin stream may be fed to the reactor from a distillation zone or unit.

According to an aspect of the present invention, an olefin-containing stream, e.g., such as native offgas, may be fed directly to the alkylation zone in the presence of an ionic liquid catalyst, where the offgas may be contacted with an isoparaffin under alkylation conditions in the presence of the ionic liquid catalyst composition to provide an alkylate product with an olefin conversion of at least about 65%.

Under the alkylation conditions within the reactor, the isoparaffin (e.g., isopentane) may be alkylated with the olefin (e.g., ethylene) to form an alkylate product suitable as gasoline blending components for producing high octane, low volatility, clean-burning gasoline.

Alkylation processes of the present invention may be biphasic. The alkylate product, together with unreacted isoparaffins, may be found in the less dense hydrocarbon phase. The more dense ionic liquid phase (catalyst) may be separated from the hydrocarbon phase in a separation zone (catalyst separator, FIG. 1). The separated ionic liquid catalyst may be recycled back to the alkylation zone. A portion of the separated catalyst, which may be partially spent or deactivated, may be fed to a regeneration zone (catalyst regeneration unit, FIG. 1) to provide reactivated catalyst, and at least a portion of the reactivated catalyst may be fed to the alkylation zone.

The alkylate product and unreacted isoparaffin(s) may be recovered separately from the hydrocarbon phase by distillation, and the latter may be recycled to the isoparaffin stream. The alkylate product may be treated as appropriate to remove any trace impurities. Any light alkanes exiting the reactor, e.g., carried by inert gas present in the olefin-containing gas stream, may be recovered, for example, by condensation, and then recycled to the reactor.

According to one aspect of the invention, an olefin-containing gas stream, e.g., offgas, containing not more than about 45 vol % ethylene, fed directly into an ionic liquid catalyst can be considered to lead to isoparaffin alkylation via a two step process. Without being bound by theory, a two step alkylation reaction involving ethylene may proceed as follows. In a first step, an alkyl halide (e.g., ethyl chloride) may be formed as an ionic liquid phase solution by contacting the ethylene-containing gas with the ionic liquid catalyst, the alkyl halide being readily soluble in the ionic liquid catalyst; and in a second step, an alkylate product may be provided by contacting the ionic liquid phase solution of alkyl halide with an isoparaffin under alkylation conditions. With respect to the second step described hereinabove, when ethyl chloride, for example, is added to acidic chloroaluminate ionic liquids, ethyl chloride reacts with AlCl₃ to form tetrachloroaluminate (AlCl₄⁻) and ethyl cation. Hydride shift from the isoparaffin (isopentane or isobutane) to the generated ethyl cation leads to the tertiary cation which propagates the inclusion of the isoparaffin in the reaction and, hence, the alkylation pathway.

It is apparent from the foregoing that processes according to the present invention enable the production of various high value gasoline blending components by using conveniently and abundantly available feedstocks, e.g., isopentane and FCC unit offgas, while avoiding expensive ethylene separation/enrichment of such offgas as performed in prior art processes (for example, using an ethylene extraction unit).

Alkylation reactions in accordance with the present invention may be conducted in one or more alkylation zones using the same or different ionic liquid catalysts. Furthermore, the invention is by no means limited to the alkylation of isopentane with ethylene. For example, according to one embodiment of the present invention, isobutane may be alkylated with ethylene to produce a high-octane C₆ gasoline blending component. Also, the olefin-containing stream may contain propylene, butylenes, and/or pentenes, which may be used for the alkylation of isoparaffins including isobutane, isopentane or their mixtures. Other variations of the instant invention may be apparent to the skilled artisan.

### EXAMPLES

The following examples are illustrative of the present invention, but are not intended to limit the invention in any way beyond what is contained in the claims which follow.

### Example 1

### Preparation of 1-Butylpyridinium Heptachlorodialuminate Ionic Liquid Catalyst

1-butylpyridinium heptachlorodialuminate is a room temperature ionic liquid prepared by mixing neat 1-butylpyridinium chloride (a solid) with neat solid aluminum trichloride in an inert atmosphere. 1-butylpyridinium chloride and the corresponding 1-butylpyridinium heptachlorodialuminate were synthesized as follows. In a 2-L Teflon-lined autoclave, 400 gm (5.05 mol.) of anhydrous pyridine (99.9% pure, Aldrich) were mixed with 650 gm (7 mol.) of 1-chlorobutane (99.5% pure, Aldrich). The neat mixture was sealed and stirred at 125° C. under autogenic pressure overnight. After cooling and venting the autoclave, the reaction mixture was diluted and dissolved in chloroform and transferred to a 3-L round bottom flask. Concentration of the reaction mixture at reduced pressure on a rotary evaporator (in a hot water bath) to remove excess chloride, unreacted pyridine, and the chloroform solvent gave a tan solid product. Purification of the product was done by dissolving the obtained solids in hot acetone and precipitating the pure product through cooling and addition of diethyl ether. Filtering and drying under vacuum and heat on a rotary evaporator gave 750 gm (88% yield) of the desired product as an off-white shiny solid. ¹H- and ¹³C-NMR were consistent with the desired 1-butylpyridinium chloride, and no impurities were observed.

1-butylpyridinium heptachlorodialuminate was prepared by slowly mixing dried 1-butylpyridinium chloride and anhydrous aluminum trichloride (AlCl₃) according to the following procedure. The 1-butylpyridinium chloride was dried under vacuum at 80° C. for 48 hours to remove residual water (1-butylpyridinium chloride is hygroscopic and readily absorbs water upon exposure to air). Five hundred grams (2.91 mol.) of the dried 1-butylpyridinium chloride were transferred to a 2-L beaker in a nitrogen atmosphere in a glove box. Then, 777.4 gm (5.83 mol.) of anhydrous powdered AlCl₃ (99.99%, Aldrich) were added in small portions (while stirring) to control the temperature of the highly exothermic reaction. Once all the AlCl₃ was added, the resulting amber liquid was left to gently stir for an additional ½-1 hour. The liquid was then filtered to remove any undissolved AlCl₃. The resulting acidic 1-butylpyridinium heptachlorodialuminate may be used as a catalyst for the alkylation of isoparaffins with olefins including ethylene.

### Example 2

### Direct Alkylation of Isoparaffin with Ethylene in Simulated Offgas

An isopentane-containing industrial isoparaffin mixture (containing 86 vol % isopentane, 9 vol % n-pentane, 4 vol % C₆₊ and 1 vol % C₄₋) was alkylated with a simulated FCC offgas containing 21 vol % ethylene and 79 vol % hydrogen (78.5 wt % ethylene and 21 wt % hydrogen). The reaction was performed in a 100 ml continuous stirred-tank reactor at a temperature of 50° C. and a pressure of 300 PSIG. Ionic liquid catalyst (1-butylpyridinium heptachlorodialuminate, Example 1) was injected at a rate of about 200 g/hr, and HCl co-catalyst was injected at a rate ranging from 0.4 to 1.1 g/hr. The isoparaffin feed rate was 75 g/hr and the offgas (olefin) feed rate was 2.2 g/hr, corresponding to an I/O (isoparaffin/olefin) molar ratio of 14 (with the exception of the data point for the olefin/HCl molar ratio of 6.75 (FIG. 2) for which the olefin feed rate was increased to 8.6 g/hr, corresponding to an I/O molar ratio of 3.7).

The ethylene conversion for the simulated offgas was in the range from about 67% to about 85% depending on the HCl flow rate (olefin/HCl molar ratio), as illustrated in FIG. 2. (For comparative purposes, the ethylene conversion in the absence of hydrogen was >95% under similar conditions.) The product consisted of predominantly a mixture of C₇-C₉ isoalkanes, with a minor C₆ component mostly derived from the isoparaffin feed. The C₁₀₊ content of the alkylate was less than 5%.

Numerous variations on the present invention are possible in light of the teachings and supporting examples described herein. It is therefore understood that within the scope of the following claims, the invention may be practiced otherwise than as specifically described or exemplified herein.

What is claimed is:

1. An alkylation process comprising: contacting in an alkylation zone under alkylation conditions an olefin containing gas stream containing not more than about 45 vol % olefins with an isoparaffin in the presence of an ionic liquid catalyst composition to provide an alkylate product.

2. The process according to claim 1, wherein the gas stream contains not more than about 35 vol % ethylene and the isoparaffin is selected from the group consisting of isopentane, isobutane, and mixtures thereof.

3. The process according to claim 1, wherein the gas stream contains not more than about 25 vol % ethylene and hydrogen gas.

4. The process according to claim 1, wherein the gas stream contains not more than about 20 vol % ethylene and at least one non-condensable gas.

5. The process according to claim 1, wherein the gas stream comprises native offgas from a fluid catalytic cracking (FCC) unit.

6. The process according to claim 1, wherein the gas stream contains at least about 50 vol % of a gas selected from the group consisting of nitrogen, methane, hydrogen, and mixtures thereof.

7. The process according to claim 1, wherein the catalyst composition comprises a chloroaluminate ionic liquid prepared from AlCl₃ and an organic halide salt selected from the group consisting of salts of the general formulas A, B, C, and D: 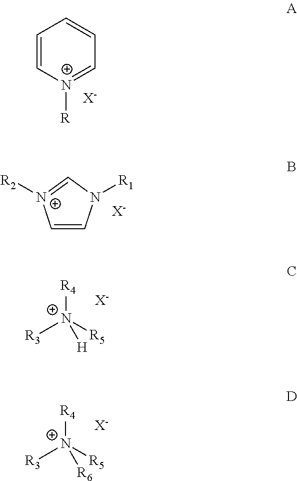
wherein R═H, methyl, ethyl, propyl, butyl, pentyl or hexyl, X is halide, each of R₁ and R₂═H, methyl, ethyl, propyl, butyl, pentyl or hexyl, wherein R₁ and R₂ may or may not be the same, each of R₃, R₄, R₅ and R₆=methyl, ethyl, propyl, butyl, pentyl or hexyl, wherein R₃, R₄, R₅ and R₆ may or may not be the same.

8. The process according to claim 1, wherein the catalyst composition comprises 1-butylpyridinium heptachlorodialuminate.

9. An alkylation process comprising: contacting in an alkylation zone under alkylation conditions an ethylene containing gas stream containing not more than about 45 vol % ethylene with an isoparaffin in the presence of a catalyst composition to provide an ethylene conversion of at least about 65%.

10. The process according to claim 9, wherein the catalyst composition comprises a chloroaluminate ionic liquid catalyst.

11. The process according to claim 9, wherein the gas stream comprises native offgas from a fluid catalytic cracking (FCC) unit.

12. The process according to claim 9, wherein the gas stream comprises unprocessed refinery offgas, and the gas stream contains not more than about 35 vol % ethylene.

13. The process according to claim 9, wherein the gas stream contains not more than about 25 vol % ethylene.

14. The process according to claim 9, wherein the gas stream contains not more than about 20 vol % ethylene at least about 55 vol % of a gas selected from the group consisting of nitrogen, methane, hydrogen, and mixtures thereof.

15. An alkylation process comprising: feeding an olefin containing native offgas into an alkylation zone in the presence of an ionic liquid catalyst composition, wherein the offgas contains not more than about 45 vol % olefins; and contacting an isoparaffin with the ionic liquid catalyst composition in the alkylation zone under alkylation conditions to provide an olefin conversion of at least about 65%.

16. The process according to claim 15, wherein the offgas comprises unprocessed offgas from a fluid catalytic cracking (FCC) unit, the offgas containing not more than about 25 vol % ethylene.

17. The process according to claim 15, wherein the offgas contains not more than about 20 vol % ethylene and at least about 55 vol % of a gas selected from the group consisting of nitrogen, methane, hydrogen, and mixtures thereof.

18. The process according to claim 15, wherein the ionic liquid catalyst comprises a chloroaluminate ionic liquid prepared from AlCl₃ and an organic halide salt selected from the group consisting of salts of the general formulas A, B, C, and D: 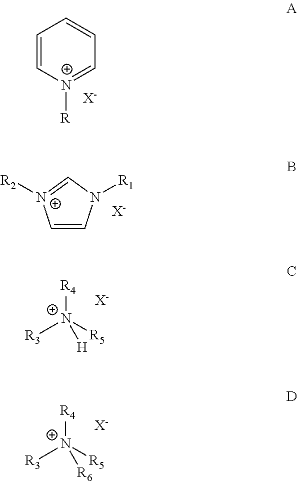
wherein R═H, methyl, ethyl, propyl, butyl, pentyl or hexyl, X is halide, each of R₁ and R₂═H, methyl, ethyl, propyl, butyl, pentyl or hexyl, wherein R₁ and R₂ may or may not be the same, each of R₃, R₄, R₅ and R₆=methyl, ethyl, propyl, butyl, pentyl or hexyl, wherein R₃, R₄, R₅ and R₆ may or may not be the same.

19. The process according to claim 15, wherein the isoparaffin is selected from the group consisting of isopentane, isobutane, and mixtures thereof.

20. The process according to claim 15, wherein the feeding step comprises contacting the offgas with the ionic liquid catalyst to provide an ionic liquid phase solution of an ethyl halide; and wherein the contacting step comprises contacting under alkylation conditions the ionic liquid phase solution of ethyl halide with the isoparaffin to provide an alkylate product.
